(19) United States
(12) Patent Application Publication
(10) Pub. No.: US 2026/0284424 A1
(43) Pub. Date: Sep. 24, 2026

(54) SYSTEMS AND METHODS FOR MANUFACTURING LIGHT THERAPY INTERFACES

(71) Applicants: RESMED PTY LTD, Bella Vista, New South Wales (AU); RESMED ASIA PTE. LTD., Singapore (SG)

(72) Inventors: Sen DAN, Singapore (SG); Garth Alan BERRIMAN, Sydney (AU)

(21) Appl. No.: 19/478,758

(22) PCT Filed: Apr. 29, 2024
(86) PCT No.: PCT/AU2024/050411

§ 371 Date: Oct. 24, 2025

Related U.S. Application Data
(60) Provisional application No. 63/498,672, filed on Apr. 27, 2023.

(51) Int. Cl. A61N 5/06

(52) U.S. Cl. CPC A61N 5/0616; A61N 5/0624; A61N 2005/0647; A61N 2005/0652; A61N 2005/0662

(57) ABSTRACT

The present invention relates to a method to manufacture a light therapy interface for a user, the method comprising: storing a facial image of the user in a storage device; determining one or more facial morphological features of the user based on the facial image; computing an interface surface from the one or more facial morphological features, the computed interface surface comprising one or more emitter coordinates each defining a spatial location on the computed interface surface for a light emitter; and generating interface fabrication instructions from the computed interface surface, the interface fabrication instructions being configured to fabricate a light therapy interface that corresponds in shape to the computed interface surface and includes an emitter incorporation zone surrounding each emitter coordinate.

### FIELD OF THE INVENTION

The present disclosure relates generally to light therapy, and more particularly, to systems and methods for manufacturing light therapy interfaces.

### BACKGROUND TO THE INVENTION

Light therapy involves exposing the skin to selected wavelengths of (typically) visible light for applications such as treating dermatological conditions (such as acne), reducing inflammation and promoting anti-aging effects. More recent light therapy techniques utilise light from light emitting diodes (LEDs) rather than from lasers. LEDs are preferable to lasers due to the potential for high-intensity laser light to cause skin damage. With this reduction in risk of skin damage, LED-based light therapy systems are now regularly sold for home use.

A typical light therapy system comprises a light therapy interface (such as a mask, veil or hood) with an array of LEDs and associated circuitry attached thereto. However, current light therapy interfaces are one-size-fits all devices, with little thought given to accommodating the individual user. This lack of customisation can have negative consequences both in terms of comfort and potentially from a therapeutic perspective.

### BRIEF DESCRIPTION OF THE DRAWINGS

FIG. 1 is a functional block diagram of a system, according to some implementations of the present disclosure.

FIG. 2A is an example facial scan that shows different landmark points to identify facial dimensions for fabricating a light therapy interface.

FIG. 2B is a view of the facial scan in FIG. 2A that shows different landmark points to identify a first facial measurement.

FIG. 2C is a view of the facial scan in FIG. 2A that shows different landmark points to identify a second facial measurement.

FIG. 2D is a view of the facial scan in FIG. 2A that shows other landmark points to identify a third facial measurement.

FIG. 3 illustrates an exemplary light therapy interface fabricated using the techniques of certain embodiments of the present disclosure.

FIG. 4 is a schematic illustration of light emitters of a light therapy interface superimposed onto the face of a user.

FIG. 5 is a flow diagram of first exemplary computing steps that, once executed, generate suitable fabrication instructions for fabricating a customised light therapy interface.

FIG. 6 is a flow diagram of second exemplary computing steps that, once executed, generate suitable fabrication instructions for fabricating a customised light therapy interface.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION OF THE DRAWINGS

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

Certain aspects and features of the present disclosure relate to manufacturing light therapy interfaces (such as masks) that are customised to the individual user. Sensor data from one or more sensors of a user device (e.g., portable user device, such as a smartphone) can be leveraged to ensure that the interface's light emitters (such as LEDs) are appropriately located relative to the user's face. The sensor(s) can collect data about the user's face and generate a face mapping therefrom that identifies one or more of the user's facial morphological features. The sensor data and face mapping can then be leveraged to compute a mathematical surface known hereafter as an ‘interface surface’. The optimal locations of light emitters are also computed from the sensor data and face mapping. These optimal locations are typically identified as spatial coordinates known hereafter as ‘emitter coordinates’. Fabrication instructions can be generated from the computed interface surface. The fabrication instructions are configured to control a suitable manufacturing process (such as additive manufacturing or milling) to fabricate a light therapy interface that is substantially congruent in shape to the shape of the interface surface. The fabrication instructions are also configured to demarcate zones in the light therapy interface where light emitters can be suitably incorporated at the desired spatial locations. These emitter incorporation zones are typically centred around the emitter coordinates.

As will be explained, the light therapy manufacturing method can be provided as a service that allows users to procure a customised light therapy interface. In one embodiment, the user uploads a facial image to a web server. The web server is in communication with computing infrastructure that executes programming to determine the user's facial morphological features, compute the interface surface and emitter incorporation zones, and generate the fabrication instructions. As discussed below, in some embodiments, the computing infrastructure includes one or more trained machine learning models that undertake some or all of the computing functions. The generated fabrication instructions are used to drive the manufacturing process to fabricate the light therapy interface. The finished light therapy interface can then be delivered to the user.

Other aspects and features of the present disclosure relate to methods and systems for generating light emitter characteristics that pertain to the light emitters of a light therapy interface. The generated light emitter characteristics can be utilised to produce a light therapy interface that delivers light therapy having specified parameters to selected locations of a user's face. This aspect of the present disclosure may utilise a therapy map to generate the light emitter characteristics. The therapy map may take the form of a suitably trained machine learning model.

According to some embodiments, the light emitter characteristics include emitter coordinates that define spatial location/s on the light therapy interface that is to be fabricated. Light emitters located at the respective spatial locations are configured to deliver light therapy to respective target locations on the user's face.

The light emitter characteristics may also include emitter densities that specify a density of light emitters present at the spatial location of each emitter coordinate.

The emitter densities can vary from spatial location to spatial location. In this way, the light emitter characteristics can define one or more spatial locations at which the light emitters will deliver more light exposure (and thus have a greater emitter density) in comparison to other spatial locations. Greater emitter density intensity results in the light emitters present at the requisite spatial location emitting light with a correspondingly greater intensity.

In another embodiment, the light emitter characteristics comprise emitter wavelengths that define one or more wavelengths of light that the light emitters emit. The treatment map may generate emitter wavelengths by reference to skin conditions or features at that location so that the emitted wavelength is appropriate to the skin conditions or features at that location.

The skin conditions or features may be determined from the received facial image. For example, the facial image may capture indications of different pigmentation around the user's eyes, where the skin is also more sensitive. The emitter wavelengths that the therapy map generates specify appropriate wavelengths and intensities to target the skin conditions and features in that region. Conditions such as acne can also be determined and located from the facial image by analysing the colour of relevant pixels or by using a suitably trained machine learning model. The emitter wavelengths that the therapy map generates for light emitters in these locations are selected to target acne, such as blue light or a combination of red and blue light.

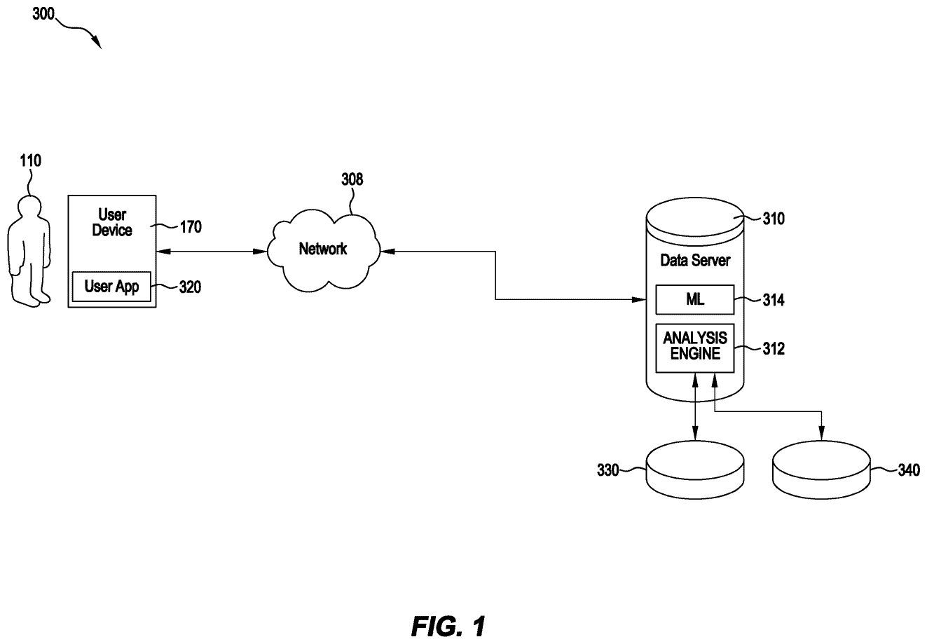

Referring to FIG. 1, a light therapy interface system 300 is illustrated. System 300 collects data that is utilised to compute (amongst other objects) an interface surface and emitter coordinates, and generate fabrication instructions for a light therapy interface that is customised for the user 110. The evaluation system 300 collects a facial image of a user 110 that the user captures by operating a user device 170.

The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The user device 170 includes a display that is generally used to display image(s) including still images, video images, or both. In some implementations, the display acts as a human-machine interface (HMI) that includes a graphic user interface (GUI) configured to display the image(s) and an input interface. The display can be an LED display, an OLED display, an LCD display, or the like. The input interface can be, for example, a touchscreen or touch-sensitive substrate, a mouse, a keyboard, or any sensor system configured to sense inputs made by a human user (such as user 110) interacting with the user device 170. In some implementations, one or more user devices can be used by and/or included in the system 300.

The above described components may be used to collect data on the user in relation to a customised light therapy interface such as an LED-equipped mask. Thus, one example of the present technology may allow users to procure a light therapy interface having LEDs in optimised locations that are customised to the user's face and therapy requirements. This potentially allows more effective light therapy. The scanning process allows a user to quickly measure their facial anatomy and segment the facial skin into different regions from the comfort of their own home using a computing device, such as a desktop computer, tablet, smart phone or other mobile device such as the user device 170. The computing device (or a remote server) may then receive the scanned facial data and generate fabrication instructions for the customised light therapy interface therefrom.

The computing device (or remote server) may also receive an image of the user's face and generate therefrom data pertaining to the characteristics of LEDs in a light therapy interface that are customised to facial skin conditions and features (such as texture, colour and the presence of rashes or lesions, or skin conditions such as acne) captured in the image.

A user app 320 installed on the user device 170 includes a user interface that instructs the user 110 in capturing a suitable facial image. The user app 320 communicates with a server 310 via a network 308. An interface evaluation engine 312 executed by the server 310 is used to process the facial image and compute the interface surface and emitter coordinates. As discussed below, the evaluation engine 312 may utilize a trained model executed by a machine learning module 314 to compute interface surfaces and emitter coordinates.

The system 300 may comprise one or more databases. The one or more databases may include a user database 330, and a light therapy interface database 340 and any other database described herein. It is to be understood that in some examples of the present technology, all data required to be accessed by a system or during performance of a method may be stored in a single database. In other examples the data may be stored in two or more separate databases. Accordingly, where there is a reference herein to a particular database, it is to be understood that in some examples the particular database may be a distinct database and in other examples it may be part of a larger database.

In some examples, the user database 330 stores facial features from a user population and a corresponding number of light therapy interfaces used by the user population. The light therapy interface database 340 stores data on different types and sizes of light therapy interfaces, such as masks. The light therapy interface database 340 may include dimensional data provided by computer aided design (CAD) data for each type of interface and for each size interface.

The analysis engine 340 includes routines for deriving a face shape from a 2D image or 3D model (whether from a 3D scanner or from converting a 2D image to a 3D model by computational means). This face shape data is utilised in computing the interface surface and locating the emitters thereon (namely by computing a set of emitter coordinates).

As will be explained, the server 310 collects the data from multiple users stored in the database 330 and corresponding mask shape data stored in the light therapy interface database 340 to evaluate whether a prefabricated mask will fit the scanned facial dimensional data collected from the new user.

As noted above, the present technology may employ a user app 320 that is downloadable from a manufacturer or third-party server to the user device 170. User app 320 includes a user interface that instructs the user 110 in operating the user device 170 to capture a suitable facial image. As instructed, the user 110 may stand in front of a mirror and press a camera button on the display of the user device 170. An activated process of the user app 320 may then take a series of pictures of the user's face, as well as obtaining facial dimensions for uploading to the server 310. This series of steps may generally be characterized as including three or four different phases: a pre-capture phase, a capture phase, a post-capture image processing phase, and a comparison and output phase.

The analysis engine 340 includes routines for identifying three-dimensional facial features from the facial images that are uploaded to the server 310. Identification of facial features is by reference to the “shape” of different facial features. The shape is described as the near continuous surface of a user's face. In reality, a continuous surface is not possible, but collecting around 10k-100k points on the face provides an approximation of the continuous surface of the face. There are several example techniques for collecting facial image data for identifying three-dimensional facial features.

One method may be determining the facial images from a 2D image. In this method, computer vision (CV) and a trained machine learning (ML) model are employed to extract key facial landmarks. For example, OpenCV and DLib libraries may be used for landmark comparison through having a trained number of standard facial landmarks. Once the preliminary facial landmarks are extracted, the derived three-dimensional features must be properly scaled. Scaling involves determining an object such as a coin, credit card or the iris of the user to provide a known scale. For example, Google Mediapipe Facemesh and Iris models may track the iris of a user and scale face landmarks. These models contain 468 landmarks of the face and 10 landmarks of the eyes. The iris data is then used to scale other identified facial features.

Another method of determining three-dimensional features may be from facial data taken from a 3D camera with a depth sensor. 3D cameras (such as that on the iphone X and above) can perform a 3D scan of a face and return a meshed (triangulated) surface. The number of surface points is generally in the order of ~50k. In this example, there are 2 types of outputs from a 3D camera such as the iPhone. These are: (a) raw scan data, and (b) a lower resolution blendshape model used for face detection and tracking. The latter includes automatic landmarking, whereas the former does not. The mesh surface data does not require scaling.

Another method is generating a 3D model directly from a 2D image. This involves using a 3D morphable model (or 3DMM) and machine learning to adapt the shape of the 3DMM to match the face in the image. Single or multiple image views are possible from multiple angles and may be derived from a video captured on a digital camera. The 3DMM may be adapted to match the data taken from the multiple 2D images via a machine learning matching routine. The 3DMM may be adapted to account for the shape, pose, and expression shown in the facial image to modify the facial features. Scaling may still be required, and thus detection and scaling of a known object such as an eye feature such as an iris could be used as a reference to account for scaling errors due to factors such as age.

The three-dimensional features or shape data may be used to compute the interface surface and the set of emitter coordinates. One way to compute an emitter coordinate is to measure a prescribed distance (such as a distance based on the expected intensity and wavelength of the light that the emitter emits) from the identified surfaces of the face.

In another embodiment of the present disclosure, emitter coordinates are computed by projecting a simulated light beam of desired intensity onto the interface surface. In this embodiment, the emitter coordinate is the location on the interface surface at which the simulated light beam contacts the interface surface. A set of emitter coordinates can be built up by projecting individual simulated light beams onto the interface surface that each originate from different locations on the user's face.

Once the set of emitter coordinates are generated, the density of LEDs at each spatial location is generated. Generally speaking, the density of LEDs at a spatial location depends on the intensity of light that the LEDs are to emit from that location and direct at the user's face.

In some cases, the user app 320 may control the user device 170 to output a visual display that includes a reference feature. The user may position the feature adjacent to their facial features, such as by movement of user device 170's in-built camera. The user device may then capture and store one or more images of the facial features in association with the reference feature when certain conditions, such as alignment conditions are satisfied. This may be done with the assistance of a mirror. The mirror reflects the displayed reference feature and the user's face to the user device 170. The user app 320 then controls the user device 170 to identify certain facial features within the images and measure distances therebetween. By image analysis processing a scaling factor may then be used to convert the facial feature measurements to a form suitable for computing the interface surface and emitter coordinates. Such form may be, for example, standardized unit of measure, such as a meter or an inch. Additional correction factors may be applied to the measurements.

In the pre-capture phase, the user app 320, among other things, assists the user 110 in establishing the proper conditions for capturing one or more images for interface surface computation. Some of these conditions include proper lighting and camera orientation and motion blur caused by an unsteady hand holding the user device 170, for example.

When the user launches the user app 320, the user app 320 may prompt the user to provide user specific information, such as age, gender, and the skin conditions sought to be treated by light therapy. However, the user app may prompt to the user to input this information at any time, such as after the user's facial features are measured. The user app 320 may also present a tutorial, which may be presented audibly and/or visually, as provided by the application to aid the user in understanding their role during the process. Also, in the pre-capture phase, the application may extrapolate the user specific information based on information already gathered by the user, such as after receiving captured images of the user's face, and based on machine learning techniques or through other artificial intelligence techniques.

When the user is prepared to proceed, which may be indicated by a user input or response to a prompt, the user app 320 activates an image sensor. The image sensor is preferably the user device's 170 forward facing camera. The camera is generally configured to capture two-dimensional images. Mobile device cameras that capture two-dimensional images are ubiquitous. The present technology takes advantage of this ubiquity to avoid burdening the user with the need to obtain specialized equipment.

Around the same time the sensor/camera is activated, the user app 320 presents a capture display on the display of the user device 170. The capture display may include a camera live action preview, a reference feature, a targeting box, and one or more status indicators or any combination thereof. In this example, the reference feature is displayed centered on the display and has a width corresponding to the width of the display. The vertical position of the reference feature may be such that the top edge of reference feature abuts the upper most edge of the display or the bottom edge of reference feature abuts the lower most edge of the display. A portion of the display will display the camera live action preview, typically showing the facial features captured by sensor/camera in real time if the user is in the correct position and orientation.

The reference feature is a feature that is known to the user app 320 and provides a frame of reference that allows the user app 320 to scale captured images. The reference feature may preferably be a feature other than a facial or anatomical feature of the user. Thus, during the image processing phase, the reference feature assists the user app 320 in determining when certain alignment conditions are satisfied, such as during the pre-capture phase. The reference features may be a quick response (QR) code or known exemplar or marker, which can provide user app certain information, such as scaling information, orientation, and/or any other desired information which can optionally be determined from the structure of the QR code. The QR code may have a square or rectangular shape. When displayed on display of the user device 170, the reference feature has predetermined dimensions, such as in units of millimeters or centimeters, the values of which may be coded into the user app 320. The actual dimensions of reference feature may vary between various computing devices. In some versions, the user app 320 may be configured to be a computing device model-specific in which the dimensions of reference feature, when displayed on the particular model, is already known. However, in other embodiments, the user app 320 may obtain certain information from the device 170, such as display size and/or zoom characteristics that allow the user app to compute the real world/actual dimensions of the reference feature as displayed on the display via scaling. Regardless, the actual dimensions of the reference feature as displayed on the display are generally known prior to post-capture image processing.

Along with the reference feature, the targeting box may be displayed on the display. The targeting box allows the user to align certain components within the field of view in the targeting box, which is desired for successful image capture.

The status indicator provides information to the user regarding the status of the process. This helps ensure the user does not make major adjustments to the positioning of the sensor/camera prior to completion of image capture.

Thus, when the user holds the display parallel to the facial features to be measured and presents the user interface to a mirror or other reflective surface, the reference feature is prominently displayed and overlays the real-time images seen by the camera/sensor and as reflected by the mirror. This reference feature may be fixed near the top of the display. The reference feature is prominently displayed in this manner at least partially so that the sensor/camera can clearly see the reference feature so that the user app 320 can easily the identify the feature to be captured. In addition, the reference feature may overlay the live view of the user's face, which helps avoid user confusion.

The user may also be instructed by the user app 320, via the display, by audible instructions via a speaker of the user device 170, or be instructed ahead of time by the tutorial, to position the display in a plane of the facial features to be measured. For example, the user may be instructed to position the display such that it is facing anteriorly and placed under, against, or adjacent to the user's chin in a plane aligned with certain facial features to be measured. For example, the display may be placed in planar alignment with the sellion and supramenton. As the images ultimately captured are two-dimensional, planar alignment helps ensure that the scale of reference feature is equally applicable to the facial feature measurements. In this regard, the distance between the mirror and both of the user's facial features and the display will be approximately the same.

When the user is positioned in front of a mirror and the display, which includes the reference feature, is roughly placed in planar alignment with the facial features to be measured, the user app 320 checks for certain conditions to help ensure sufficient alignment. One exemplary condition that may be established by the application, as previously mentioned, is that the entirety of the reference feature must be detected within a targeting box in order to proceed. If the user app 320 detects that the reference feature is not entirely positioned within the targeting box, the user app may prohibit or delay image capture. The user may then move their face along with the display to maintain planarity until the reference feature, as displayed in the live action preview, is located within the targeting box. This helps optimized alignment of the facial features and the display with respect to the mirror for image capture.

When the user app 320 detects the entirety of reference feature within the targeting box, the user app may read an inertial motion unit (IMU) of the user device 170 for detection of device tilt angle. The IMU may include an accelerometer or gyroscope, for example. Thus, the user app 320 may evaluate device tilt such as by comparison against one or more thresholds to ensure it is in a suitable range. For example, if it is determined that the user device 170, and consequently the display and user's facial features, is tilted in any direction within about ±5 degrees, the process may proceed to the capture phase. In other embodiments, the tilt angle for continuing may be within about ±10 degrees, ±7 degrees, ±3 degrees, or ±1 degree. If excessive tilt is detected a warning message may be displayed or sounded to correct the undesired tilt. This is particularly useful for assisting the user to help prohibit or reduce excessive tilt, particularly in the anterior-posterior direction, which if not corrected, could pose as a source of measuring error as the captive reference image will not have a proper aspect ratio. In some embodiments, an algorithm which accounts for tilt may be used so that the reconstruction is less sensitive to excessive tilt.

When alignment has been determined by the user app 320, the user app 320 proceeds into the capture phase. The capture phase preferably occurs automatically once the alignment parameters and any other conditions precedent are satisfied. However, in some embodiments, the user may initiate the capture in response to a prompt to do so.

When image capture is initiated, the user device 170 via its inbuilt sensor captures a number n of images, which is preferably more than one image. For example, the user device 170 may capture about 5 to 20 images, 10 to 20 images, or 10 to 15 images, etc. The quantity of images captured may be sequential such as a video. In other words, the number of images that are captured may be based on the number of images of a predetermined resolution that can be captured by sensor/camera during a predetermined time interval. For example, if the number of images sensor/camera can capture at the predetermined resolution in 1 second is 40 images and the predetermined time interval for capture is 1 second, a sensor will capture 40 images for processing with a processor of user device 170. The quantity of images may be user-defined, determined by artificial intelligence or machine learning of environmental conditions detected, or based on an intended accuracy target. For example, if high accuracy is required then more captured images may be required. Although, it is preferable to capture multiple images for processing, one image is contemplated and may be successful for use in obtaining accurate measurements. However, more than one image allows average measurements to be obtained. This may reduce error/inconsistencies and increase accuracy. The images may be placed by the user device 170 in its local memory for post-capture processing.

In addition, accuracy may be enhanced by images from multiple views, especially for 3D facial shapes. For such 3D facial shapes, a front image, a side profile and some images in between may be used to capture the face shape. When combining landmarks from multiple views, averaging can be done, but averaging suffers from inherent inaccuracy. Some uncertainty is assigned to landmark location, and landmarks are then weighted by uncertainty during reconstruction. For example, landmarks from a frontal image will be used to reconstruct the front part of the face, and landmarks from profile shots will we used to reconstruct the sides of the head. Typically, the images will be associated with the pose of the head (angles of rotation). In this manner, it is ensured that a number of images from different views are captured. For example, if eye iris is used as the scaling features, then images where the iris is closed (e.g., when the user blinks) need to be discarded as they cannot be scaled. This is another reason to require multiple images as certain images that may not be useful may be discarded without requesting rescan.

Once the images are captured, the images are processed by the user app to detect or identify facial features/landmarks and measure distances between landmarks. The resultant measurements may be used to compute an interface surface that is substantially congruent in shape to that of the user's face. This processing may alternatively be performed by an external device such as a server receiving the transmitted captured images. Processing may also be undertaken by a combination of the user app 320 and an external device.

The user app 320 retrieves one or more captured images from local memory. The image is then extracted by the user app to identify each pixel comprising the two-dimensional captured image. The user app 320 then detects certain pre-designated facial features within the pixel formation.

Detection may be performed by the user app 320 using edge detection, such as Canny, Prewitt, Sobel, or Robert's edge detection, and more advanced deep neural networks (DNNs) such as Convolutional Neural Networks (CNNs) based methods for example. These edge detection techniques/algorithms help identify the location of certain facial features within the pixel formation, which correspond to the actual facial features as presented for image capture. For example, the edge detection techniques can first identify the user's face within the image and also identify pixel locations within the image corresponding to specific facial features, such as each eye and borders thereof, the mouth and corners thereof, left and right alares, sellion, supramenton, glabella and left and right nasolabial sulci, etc. The user app 320 may then mark, tag or store the particular pixel location(s) of each of these facial features. Alternatively, or if such detection by the user app 320 is unsuccessful, the pre-designated facial features may be manually detected and marked, tagged or stored by a human operator with viewing access to the captured images through a user interface of the user device 170.

Once the pixel coordinates for these facial features are identified, the user app 320 measures the pixel distance between certain of the identified features. For example, the distance may generally be determined by the number of pixels for each feature and may include scaling. For example, measurements between the left and right alares may be taken to determine pixel width of the nose and/or between the sellion and supramenton to determine the pixel height of the face. Other examples include pixel distance between each eye, between mouth corners, and between left and right nasolabial sulci to obtain additional measurement data of particular structures like the mouth. Further distances between facial features can be measured. In this example, certain facial dimensions are used to compute the interface surface and locate the visible light emitters thereon.

Other methods for facial identification may be used. For example, fitting of 3D morphable models (3DMMs) to the 2D images using DNNs may be employed. The end result of such DNN methods is a full 3D surface (comprised of thousands of vertices) of the face, ears and head that may all be predicted from a single image or multiple multi-view images. Differential rendering, which involves using photometric loss to fit the model, may be applied. This minimizes the error (including at a pixel level) between a rendered version of the 3DMM and the image.

Once the pixel measurements of the pre-designated facial features are obtained, an anthropometric correction factor(s) may be applied to the measurements. It should be understood that this correction factor can be applied before or after applying a scaling factor, as described below. The anthropometric correction factor can correct for errors that may occur in the automated process, which may be observed to occur consistently from user to user. In other words, without the correction factor, the automated process, alone, may result in consistent results from user to user, but results that may lead to a certain amount of inaccuracy in capturing facial morphological features. This correction factor can be refined or improved in accuracy over time as measurements of each user are communicated from respective user devices to a server where such data may be further processed.

In order to apply the facial feature measurements to interface surface computation, whether corrected or uncorrected by the anthropometric correction factor, the measurements may be scaled from pixel units to other values that accurately reflect the distances between the user's facial features as presented for image capture. The reference feature may be used to obtain a scaling value or values. Thus, the user app 320 similarly determines the reference feature's dimensions, which can include pixel width and/or pixel height (x and y) measurements (e.g., pixel counts) of the entire reference feature. More detailed measurements of the pixel dimensions of the many squares/dots that comprise a QR code reference feature, and/or pixel area occupied by the reference feature and its constituent parts may also be determined. Thus, each square or dot of the QR code reference feature may be measured in pixel units to determine a scaling factor based on the pixel measurement of each dot and then averaged among all the squares or dots that are measured, which can increase accuracy of the scaling factor as compared to a single measurement of the full size of the QR code reference feature. However, it should be understood that whatever measurements are taken of the reference feature, the measurements may be utilized to scale a pixel measurement of the reference feature to a corresponding known dimension of the reference feature.

Once the measurements of the reference feature are taken by the user app 320, the scaling factor is calculated. The pixel measurements of reference features are related to the known corresponding dimensions of the reference feature, e.g., the reference feature as displayed by the display for image capture, to obtain a conversion or scaling factor. Such a scaling factor may be in the form of length/pixel or area/pixel. In other words, the known dimension(s) may be divided by the corresponding pixel measurement(s) (e.g., count(s)).

The user app 320 then applies the scaling factor to the facial feature measurements (pixel counts) to convert the measurements from pixel units to other units to reflect distances between the user's actual facial features suitable for computing an interface surface and emitter coordinates. This may typically involve multiplying the scaling factor by the pixel counts of the distance(s) for facial features that serve as reference points for locating the light emitters on a light therapy mask.

These measurement steps and calculation steps for both the facial features and reference feature are repeated for each captured image until each image in the set has facial feature measurements that are scaled and/or corrected.

The corrected and scaled measurements for the set of images may then optionally be averaged or weighted by some statistical measure such as uncertainty to obtain final measurements of the user's facial anatomy. Such measurements may reflect distances between the user's facial features.

In the comparison and output phase, results from the post-capture image processing phase may be directly output (displayed) to a person of interest or uploaded directly to the server to procure the customized light therapy interface.

In a further embodiment, the final facial feature measurements that reflect the distances between the actual facial features of the user are compared by the analysis engine 312 to light therapy interface size data such as in the light therapy interface database 340. The data record may be part of the application for automatic facial feature measurements and light therapy interface sizing. This data record can include, for example, a lookup table accessible by the analysis engine 312, which may include user interface sizes corresponding to a range of facial feature distances/values. Multiple tables may be included in the data record, many of which may correspond to a particular form of light therapy interface.

The example process for computing the interface surface and emitter coordinates identifies key landmarks from the facial image captured by the above mentioned method. In this example, the emitter coordinates are calculated by reference to facial landmarks that coincide with the skin concerns that the light therapy interface is configured to target. For example, when the light therapy interface is configured to target areas of the skin with fine lines and wrinkles, the emitter coordinates are computed to coincide with facial landmarks that have a higher probability of including such features (for example the area adjacent to the user's eyes).

Alternatively, other data relating to facial 3D shapes may also be used. For example, landmarks and any area of the face can be obtained by fitting a 3D morphable model (3DMM) onto a 3D face scan of a user. This fitting process is also known as non-rigid registration or (shrink) wrapping. Once a 3DMM is registered to a 3D scan, the emitter coordinates may be determined using any number of methods, as the points and surface of the user's face are all known.

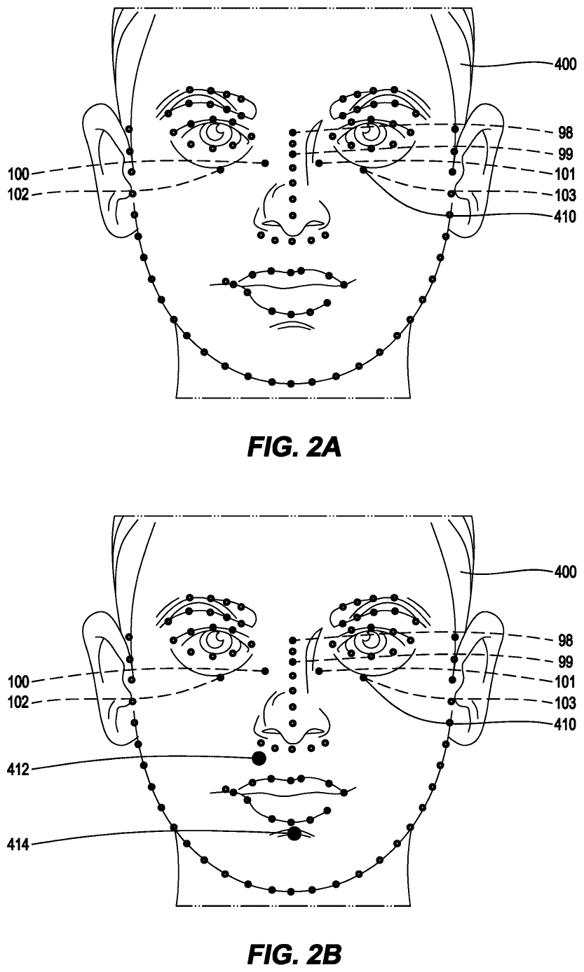

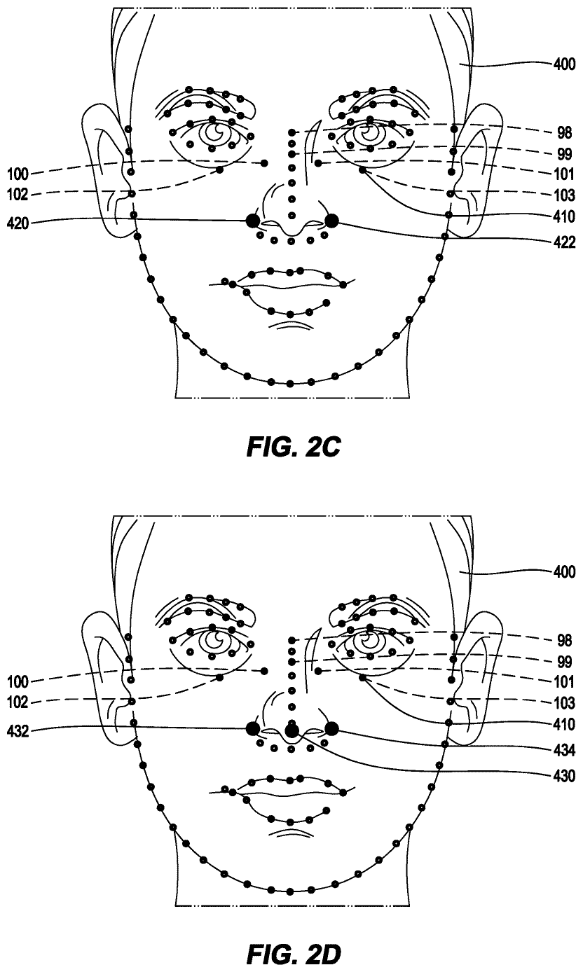

FIG. 2A is a facial image 400 such as one captured by the user app 320 described above that may be used for determining the face height dimension, the nose width dimension and the nose depth dimension. The image 400 includes a series of landmark points 410 that may be determined from the image 400 via any standardly known method. In this example, there are Standard landmark sets Open CV (68 landmark points) and some other landmarks specific to mask sizing e.g. nose, below mouth, etc. that are identified and shown on the facial image 400. In this example, the method requires seven landmarks on the facial image to determine the face height, nose width and nose depth, for mask sizing relating to the users. As will be explained, two existing landmarks may be used. The location of the three dimensions requires five additional landmarks to be identified on the image via the processing method. Based on the imaging data and/or existing landmarks, new landmarks may be determined. The two existing landmarks that will be used include a point on the sellion (nasal bridge) and a point on the nose tip. The five new landmarks required include a point on the supramenton (top of the chin), left and right alar points, and left and right alar-facial groove points.

FIG. 2B shows the facial image 400 where the face height dimension (sellion to supramenton) is defined via landmark points 412 and 414. The landmark 412 is an existing landmark point on the sellion. The landmark point 414 is a point on the supramenton. The face height dimension is determined from the distance between the landmark points 412 and 414.

FIG. 2C shows the facial image 400 with new landmark points 420 and 422 to locate the nose width dimension. This requires two new landmarks, one on each side of the nose. These are called the right and left alar points and may correspond to the right and left alare. The distance between these points provides the nose width dimension. The alar points are different, but similar to, the alar-facial groove points.

FIG. 2D shows the facial image 400 with landmark points 430, 432 and 434 to determine the nose depth dimension. A suitable landmark is available for the landmark point 430 at the nose tip. The landmark points 432 and 434 are determined at the left and right sides of the nose. The landmark points 432 and 434 are at alar-facial grooves on the left and right sides of the nose. These are similar to alar points but at the back of the nose. The examples are only one of many ways to define a plurality of landmarks. Other methods may result in more accurate estimations of anatomical measurements by using dense landmarks around regions of interest.

Machine learning may be applied to optimize the light therapy interface procurement process based on training a neural network on training data including facial images, facial morphological features, interface surfaces and emitter coordinates. Such machine learning may be executed by the server 310. The interface surface and emitter coordinate computation algorithms may be trained with a training data set based on the outputs of favourable operational results such as light therapy interfaces with light emitters in optimised locations for efficient light therapy treatment. Machine learning may be used to discover correlation between the location of light emitters on the light therapy interface and predictive inputs such as facial dimensions and other facial morphological features. Machine learning may employ techniques such as neural networks, clustering or traditional regression techniques. Test data may be used to test different types of machine learning algorithms and determine which one has the best accuracy in relation to predicting correlations.

The model for computing interface surfaces and emitter coordinates may be continuously updated by new input data from the system in FIG. 1. Thus, the model may become more accurate with greater use of the light therapy interface procurement tool by more users.

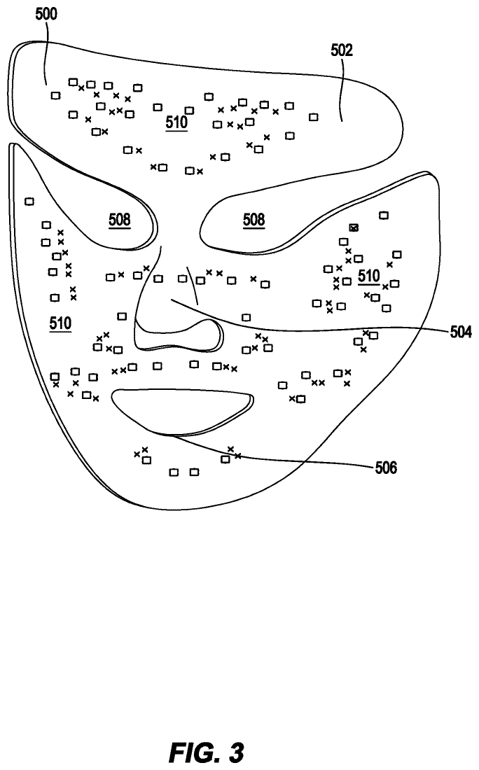

The example application that may be executed on the user device 170 in FIG. 1 or the server 310 in FIG. 3, computes the interface surface and emitter coordinates using facial morphological features determined from one or more facial images of the user. For example, an application that computes emitter coordinates based on facial landmarks may output an interface surface with the same shape as the user's face and emitter coordinates that coincide with selected facial landmarks when the user wears a light therapy interface fabricated from the interface surface.

The interface surface and emitter coordinates in this example are determined by the machine learning module 314 in FIG. 1. The example machine learning model of the machine learning module 314 is trained data including facial images with facial morphological features, interface surfaces and emitter coordinates.

FIG. 3 shows a light therapy interface 500 in the form of a mask fabricated using the present disclosure. Light therapy interface 500 is typically fabricated from a silicon material and comprises an inner surface 502 and an outer surface (not shown). The shape of the inner surface 502, which incorporates regions for the nose 504, mouth 506 and eyes 508, is defined numerically rather than algebraically. The set of points on the surface (namely the interface surface) are computed from the facial scan data using the techniques discussed above.

Light therapy interface 500 includes a plurality of LEDs 510 placed at different spatial locations throughout the inner surface 502. In the illustrated embodiment, the LEDs 510 are located in the two cheek regions and forehead regions of the inner surface 500. The inner surface 500 functions as a base layer for the light therapy interface 500. Although not illustrated in FIG. 3, light therapy interface also includes a foam layer attached to the outer surface of the light therapy interface 500 and a structural layer (formed from a semi-rigid plastic material) attached to the foam layer.

The LEDs 510 are adjustable so that the intensity of the therapeutic light is tailored to characteristics of the user 110's skin. For example, LEDs 510 are adjustable to deliver light of predetermined intensity to match the user's pigment level and age. Other characteristics of the user's skin can be used as a basis for adjusting the intensity of the light delivered by LEDs 510.

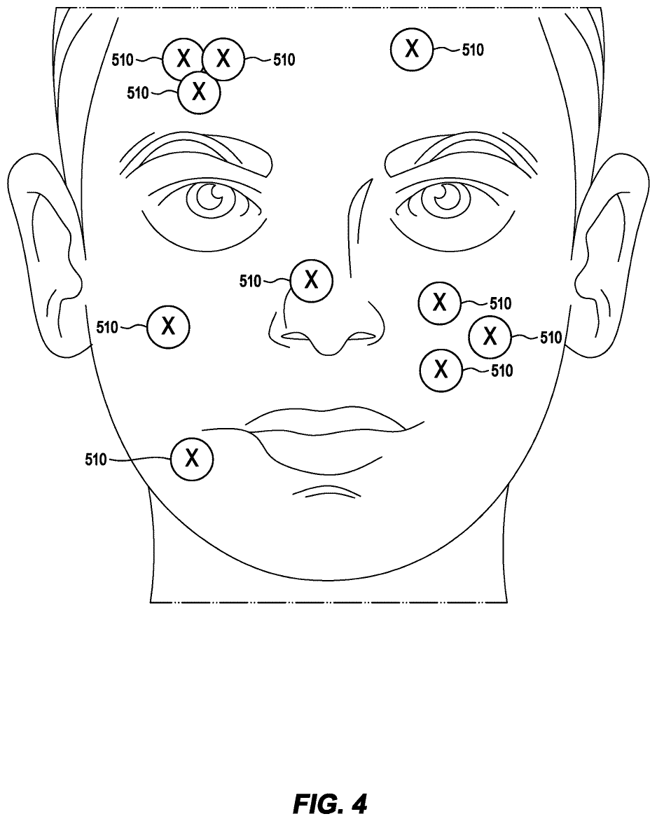

FIG. 4 is a schematic illustration of the LEDs 510 of light therapy interface 500 superimposed onto the user's face. The superimposed LEDs 510 schematically illustrate the delivery of light therapy to different locations on the user's face when the user is wearing the light therapy interface 500. Each LED 510 (shown with an ‘x’ in FIG. 4) is positioned at a spatial location on the light therapy interface 500 and emits light to a coinciding location on the user's face. The emitter incorporation zones that surround each LED 500 are shown as circles in FIG. 4.

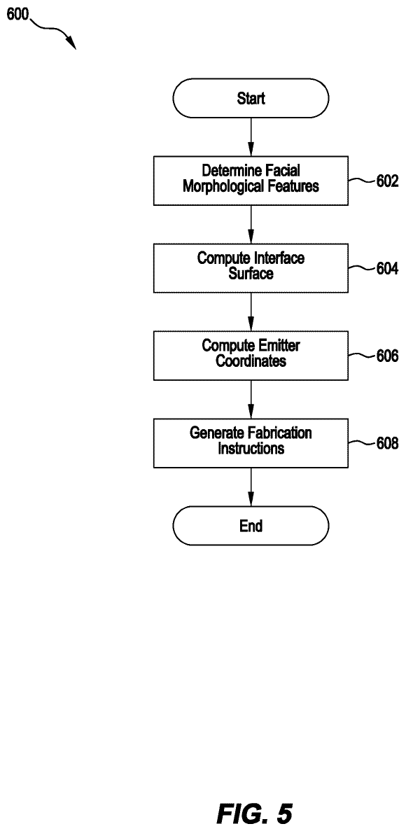

The operation of the example analysis engine 312 shown in FIG. 1, which may be controlled on the example server and a user device 170, will now be described with reference to FIG. 1 in conjunction with the flow diagram shown in FIG. 5. The flow diagram in FIG. 5 is representative of example machine readable instructions for implementing the application to fabricate a customised light therapy interface for a specific face of a user. In this example, the machine readable instructions comprise an algorithm for execution by: (a) a processor, (b) a controller, and/or (c) one or more other suitable processing device(s). The algorithm may be embodied in software stored on tangible media such as, for example, a flash memory, a CD-ROM, a floppy disk, a hard drive, a digital video (versatile) disk (DVD), or other memory devices, but persons of ordinary skill in the art will readily appreciate that the entire algorithm and/or parts thereof could alternatively be executed by a device other than a processor and/or embodied in firmware or dedicated hardware in a well-known manner (e.g., it may be implemented by an application specific integrated circuit (ASIC), a programmable logic device (PLD), a field programmable logic device (FPLD), a field programmable gate array (FPGA), discrete logic, etc.). For example, any or all of the components of the interfaces could be implemented by software, hardware, and/or firmware. Further, although the example algorithm is described with reference to the flowcharts illustrated in FIG. 5, persons of ordinary skill in the art will readily appreciate that many other methods of implementing the example machine readable instructions may alternatively be used. For example, the order of execution of the blocks may be changed, and/or some of the blocks described may be changed, eliminated, or combined.

In this example, programming executing on server 310 determines facial morphological features from one or more facial images using the techniques discussed above (602). The programming then computes an interface surface (604) and a set of emitter coordinates (606). The programming then generates fabrication instructions (608) from the interface surface and emitter coordinates.

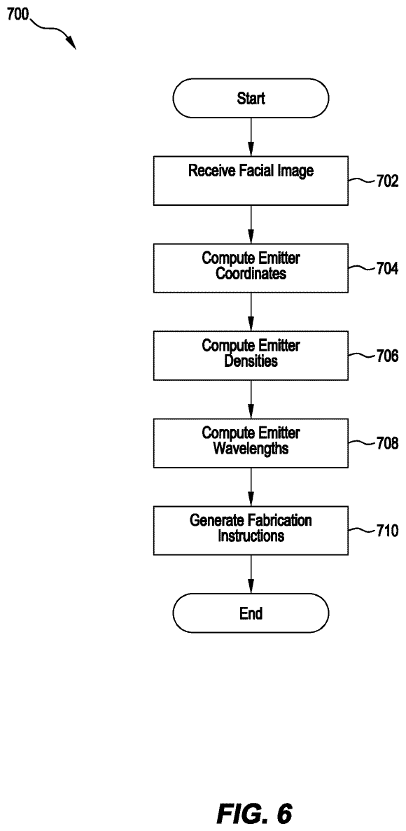

Another example of the operation of the exemplified analysis engine 312 shown in FIG. 1, will now be described with reference to FIG. 1 in conjunction with the flow diagram shown in FIG. 6. The flow diagram in FIG. 5 is representative of example machine readable instructions for implementing the application to fabricate a customised light therapy that delivers light therapy having specified parameters to selected locations of the user's face.

In this example, at step 702 programming executing on server 310 receives an image of the user 110's face that the user captures with the camera on the user device 170 (702).

At step 704, after performing any necessary pre-processing of the received image, the programming submits the received image to the machine learning module 314. In this embodiment, the machine learning module 314 includes a trained machine learning model that implements a therapy map. The therapy map is produced by training a suitable neural network on a dataset of facial images that are labelled with skin conditions and features and emitter characteristics. The therapy map performs inferencing on the received image and thus computes the emitter coordinates.

At step 706, the therapy map performs inferencing on the received image and thus computes the emitter densities.

At step 708, the therapy map performs inferencing on the received image and thus generates the emitter wavelengths.

At step 710, the programming generates fabrication instructions from the computed emitter coordinates, emitter densities and emitter wavelengths.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method to manufacture a light therapy interface for a user, the method comprising:
storing a facial image of the user in a storage device;
determining one or more facial morphological features of the user based on the facial image;
computing an interface surface from the one or more facial morphological features, the computed interface surface comprising one or more emitter coordinates each defining a spatial location on the computed interface surface for a light emitter; and
generating interface fabrication instructions from the computed interface surface, the interface fabrication instructions being configured to fabricate a light therapy interface that corresponds in shape to the computed interface surface and includes an emitter incorporation zone surrounding each emitter coordinate.

2. A method according to claim 1, wherein the or each spatial location is computed from the one or more facial morphological features.

3. A method according to claim 2, wherein the or each spatial location is computed to coincide with one or more facial morphological features when the user is wearing the fabricated light therapy interface.

4. A method according to claim 3, wherein the facial morphological features comprise the user's face contours, eyes, nose, mouth, cheeks, chin and ears

5. A method according to claim 1, further comprising computing a respective light emitter characteristic for one or more light emitters that are to be incorporated into the fabricated light therapy interface at an emitter incorporation zone.

6. A method according to claim 5, wherein the light emitter characteristics are computed from the facial image.

7. A method according to claim 6, wherein the light emitter characteristics are computed by:
processing the facial image to identify one or more regions of the face each having a specified skin characteristic; and
computing the light emitter characteristic to accord with the specified skin characteristic.

8. A method according to claim 7, wherein computing the light emitter characteristics comprises inputting the processed facial image to a therapy map, the therapy map including mappings of specified skin characteristics to light emitter characteristics.

9. A method according to claim 8, wherein the therapy map comprises a machine learning model trained to output light emitter characteristics from an input facial image that includes one or more specified skin characteristics.

10. A method according to claim 5, wherein the light emitter characteristic comprises an emitter density that specifies a density of light emitters present at the spatial location.

11. A method according to claim 10, wherein the emitter densities vary from spatial location to spatial location.

12. A method according to claim 5, wherein the light emitter characteristic comprises emitter wavelengths that define one or more wavelengths of light that the light emitter emits.

13. A method according to claim 7, wherein the specified skin characteristics comprise the location of the region on the user's face, and a skin condition.

14. A method according to claim 13, wherein the skin condition includes acne.

15. A method according to claim 1, further comprising:
training a neural network with a dataset of facial images with facial morphological features, interface surfaces and spatial locations to generate a machine learning model;
inputting an input facial image to the machine learning model; and
receiving an output interface surface with one or more emitter incorporation zones from the machine learning model.

16. A non-transitory computer-readable storage medium storing one or more programs, the one or more programs comprising instructions, which when executed by one or more processors of an electronic device, cause the electronic device to:
determine one or more facial morphological features of a user from a facial image of the user;
compute an interface surface from the one or more facial morphological features, the computed interface surface comprising one or more emitter coordinates each defining a spatial location on the computed interface surface of a light emitter; and
generate interface fabrication instructions from the computed interface surface, the interface fabrication instructions being configured to fabricate a light therapy interface that corresponds in shape to the computed interface surface and includes an emitter incorporation zone surrounding each emitter coordinate.

17. A light therapy interface manufacturing system comprising:
a light therapy interface procurement application installed on a user device, the light therapy interface procurement application including computer program code which when executed by a processor of the user device causes the user device to capture one or more images of the user's face and upload the one or more images to a light therapy interface procurement server; and
a light therapy interface procurement server, the light therapy interface procurement server including computer program code which when executed by a processor of the light therapy interface procurement server, causes the light therapy interface procurement server to:
receive the one or more images from the light therapy interface procurement application;
determine one or more facial morphological features of a user from the one or more images;
compute an interface surface from the one or more facial morphological features, the computed interface surface comprising one or more emitter coordinates each defining a spatial location on the computed interface surface for a visible light emitter; and
generate interface fabrication instructions from the computed interface surface, the interface fabrication instructions being configured to fabricate a light therapy interface that corresponds in shape to the computed interface surface and includes an emitter incorporation zone surrounding each emitter coordinate.

18. A method to manufacture a light therapy interface for a user, the method comprising:
storing a facial image of the user in a storage device;
determining one or more light emitter characteristics based on the facial image, the light emitter characteristics pertaining to light emitters of a light therapy interface that is to be fabricated; and
generating interface fabrication instructions from the light emitter characteristics, the interface fabrication instructions being configured to fabricate a light therapy interface that includes light emitters that have the light emitter characteristics.

19. A method of claim 18, wherein determining the one or more light emitter characteristics comprises inputting the facial image to a therapy map, the therapy map including mappings of skin features to light emitter characteristics.

20. A method according to claim 19, wherein the therapy map comprises a machine learning model trained to output light emitter characteristics from a facial image that includes one or more skin features.

21. A method according to claim 20, wherein the light emitter characteristics comprise emitter coordinates that define spatial location/s on the light therapy interface that is to be fabricated.

22. A method according to claim 20, wherein the light emitter characteristics comprise emitter densities that specify a density of light emitters present at the spatial location of each emitter coordinate.

23. A method according to claim 22, wherein the emitter densities vary from vary from spatial location to spatial location.

24. A method according to claim 18, wherein the light emitter characteristics comprise emitter wavelengths that define one or more wavelengths of light that the light emitters emit.

25. (canceled)
